(19) United States
(12) Patent Application Publication
(10) Pub. No.: US 2018/0200322 A1
(43) Pub. Date: Jul. 19, 2018

(54) Arthritis Relief and Remedy Method

(71) Applicant: Min Luo, State College, PA (US)

(72) Inventor: Min Luo, State College, PA (US)

(21) Appl. No.: 15/409,752

(22) Filed: Jan. 19, 2017

(51) Int. Cl. A61K 36/9068; A61K 36/282

(52) U.S. Cl. CPC A61K 36/9068; A61B 18/06; A61K 36/282

(57) ABSTRACT

A method for relieving the symptoms of arthritis externally is described. The method involves the use of sliced ginger root, moxa, produced from mugwort, and a needle. Freshly cut ginger roots are sliced into slices approximately one centimeter thick and placed on the skin near the area afflicted with arthritis. The moxa, preferably in the form of a rolled moxa cigar, is stood vertically atop the slice of ginger and ignited. The moxa cigar is permitted to burn down to the surface of the slice of ginger, heating the ginger as well as the skin of the patient. Due to the heat, the skin of the patient is blistered. After a duration of time, the ginger slice is removed from the skin, the blister is punctured with a needle, and allowed to heal. This process leaves the patient free of arthritis-induced pain or similar symptoms.

### FIELD OF THE PRESENT INVENTION

The present invention relates generally to methods configured to remedy pain, and more specifically relates to a method of relieving arthritis pain and symptoms via a natural process employing moxibustion and ginger.

### BACKGROUND OF THE PRESENT INVENTION

Numerous individuals world-wide suffer from various forms of arthritis. Unfortunately, the primary symptoms of arthritis include pain, tension, and discomfort associated with the joints of the body. Many individuals experience arthritis pain in the hands, knees, ankles, wrists, or at or near other primary joints on the body. The pain often prevents these individuals from performing a wide variety of activities, including walking, running, typing, playing catch, using fine motor skills, writing, gardening, and other common activities.

Many medications have been crafted to attempt to relieve the pain caused by arthritis. For example, some creams claim to relieve the symptoms of arthritis, as do some oral medications. While some creams are effective in relieving the pain of arthritis, the relief is always temporary, and additional application of the cream is required for the pain to subside again. Likewise, many individuals find that oral medication does not work well, or simply does not work well enough to warrant enduring a prohibitive side effect of the oral medication. As such, many of these individuals still struggle with the pain of arthritis daily.

If there were an easy way that individuals could naturally be relieved of arthritis pain for a long period of time without the use of expensive oral medications, sufferers of arthritis could be pain-free and able to enjoy their favorite activities without struggling with arthritis.

Thus, there is a need for a process that relieves the pain and discomfort caused by arthritis that does not require surgery or expensive medication that instead employs a method of moxification over ginger slices to relieve the pain.

### SUMMARY OF THE PRESENT INVENTION

The present invention is a method for remedying the pain and discomfort conventionally associated with various forms of arthritis, such as Rheumatoid Arthritis. The method of the present invention involve the procurement and specific placement of raw ginger root slices onto the skin at or near the arthritis symptoms experienced by an individual (patient). Moxibustion is then applied to the top of the ginger root via a moxa cigar. The moxa cigar, composed of ground and dried mugwort, is carefully stood up vertically on the ginger slice, which is resting on the skin of the patient. Then, the moxa cigar is ignited, and proceeds to burn slowly. An ember of the moxa cigar progressively burns down the cigar towards the ginger slice.

As the ember present on the burning moxa approaches the ginger, the skin of the patient is blistered. Once the moxa cigar has been completely burnt and extinguished, the ginger and moxa ash is removed from the skin of the patient. Finally, an acupuncture needle is sterilized and then employed to puncture the blister, venting the liquid housed within the blister, relieving the arthritis pain of the patient.

### BRIEF DESCRIPTION OF THE DRAWINGS

The present invention will be better understood with reference to the appended drawing sheets, wherein:

FIG. 1 is a flow chart depicting the preferred process of use of the present invention.

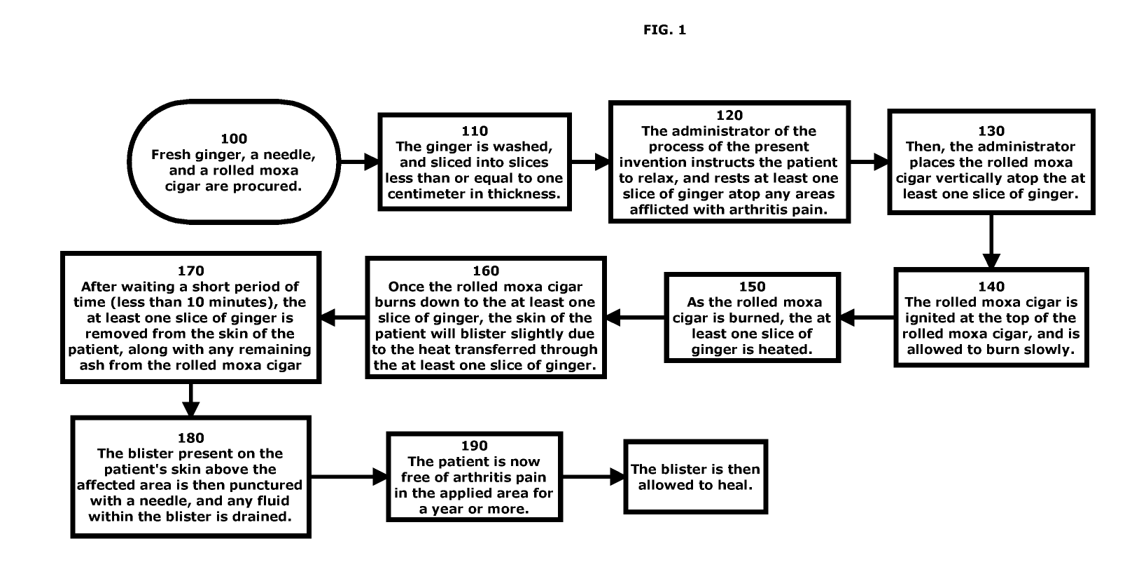

FIG. 2 is a view of the process of the present invention in use on the skin of a knee of an individual.

### DETAILED DESCRIPTION OF THE PREFERRED EMBODIMENT

The preferred embodiment of the method of the present invention involves the use of a moxa cigar (10) which is preferably a rolled, finely ground and dried mugwort. Additionally, ginger (20) is employed. The ginger (20) is preferably freshly cut, and is preferably washed prior to use. The ginger (20) is cut into slices approximately one centimeter in thickness or less for use in the method of the present invention.

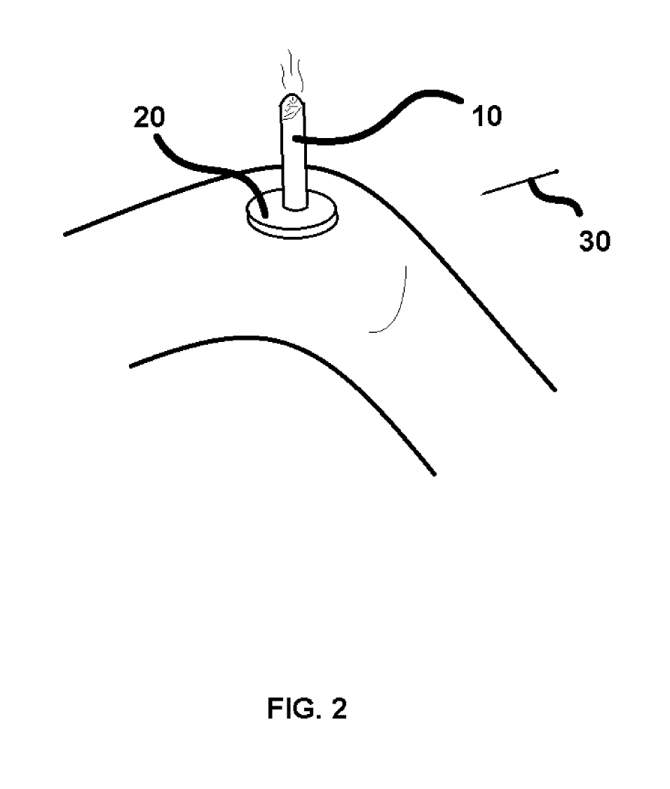

The process of use of the present invention, as depicted in FIG. 2, is preferably as follows:
1. Fresh ginger, a needle (30), and a rolled moxa cigar are procured. (100)
2. The ginger is washed, and sliced into slices less than or equal to one centimeter in thickness. (110)
3. The administrator of the process of the present invention instructs the patient to relax, and rests at least one slice of ginger atop any areas afflicted with arthritis pain. (120)
4. Then, the administrator places the rolled moxa cigar vertically atop the at least one slice of ginger. (130)
5. The rolled moxa cigar is ignited at the top of the rolled moxa cigar, and is allowed to burn slowly. (140)
6. As the rolled moxa cigar is burned, the at least one slice of ginger is heated. (150)
7. Once the rolled moxa cigar burns down to the at least one slice of ginger, the skin of the patient will blister slightly due to the heat transferred through the at least one slice of ginger. (160)
8. After waiting a short period of time (less than 10 minutes), the at least one slice of ginger is removed from the skin of the patient, along with any remaining ash from the rolled moxa cigar. (170)
9. The blister present on the patient's skin above the affected area is then punctured with a needle (30), and any fluid within the blister is drained. (180) The needle (30) is preferably sterilized with isopropyl alcohol prior to use.
10. The patient is now free of arthritis pain in the applied area for a year or more. (190)
11. The blister is then allowed to heal. (200)

Alternate embodiments of the present invention include variations on the thickness of the ginger root slices, as well as variations on the size, shape, and length of the moxa cigar (10) employed in the process of the present invention.

It should be noted that fresh ginger (20) is preferably used, rather than frozen or old ginger. Ideally, the ginger (20) is approximately the same thickness of 15 stacked sheets of paper. If the ginger (20) is cut too thin, not enough juice is supplied by the ginger (20). If the ginger (10) is too thick, not enough heat from the moxa cigar (10) is available to provide enough heat to create the blister. The moxa cigar (10) is intended to burn naturally until it burns out. The blister is preferably punctured with the needle (30) immediately after it forms. The blister can be sealed with Vasoline™ or similar petroleum jelly.

Having illustrated the present invention, it should be understood that various adjustments and versions might be implemented without venturing away from the essence of the present invention. Further, it should be understood that the present invention is not solely limited to the invention as described in the embodiments above, but further comprises any and all embodiments within the scope of this application.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The exemplary embodiment was chosen and described in order to best explain the principles of the present invention and its practical application, to thereby enable others skilled in the art to best utilize the present invention and various embodiments with various modifications as are suited to the particular use contemplated.

I claim:

1. A method for relieving symptoms of arthritis from a patient comprising:
slicing a ginger root into at least one slice of ginger root approximately one centimeter in thickness;
applying the at least one slice of ginger root to an area of skin of the patient proximal to symptoms of arthritis;
placing a moxa cigar atop the at least one slice of ginger root;
igniting the moxa cigar;
burning the entirety of the moxa cigar, allowing the ginger root to be heated;
the at least one slice of ginger root blistering skin of the patient;
puncturing the blister;
draining contents of the blister; and
allowing the blister to heal.
